(19) United States
(12) Patent Application Publication
(10) Pub. No.: US 2026/0256837 A1
(43) Pub. Date: Sep. 3, 2026

(54) Palatability-Enhanced Activated Charcoal Systems Enabling Voluntary Ingestion for Toxin Removal in Animals

(71) Applicant: Pet Vet Products, LLC, San Clemente, CA (US)

(72) Inventor: Leah Hill, San clemente, CA (US)

(73) Assignee: Pet Vet Products, LLC, San clemente, CA (US)

(21) Appl. No.: 19/651,739

(22) Filed: Apr. 19, 2026

Related U.S. Application Data
(60) Provisional application No. 63/905,043, filed on Oct. 24, 2025.

(51) Int. Cl. A61K 33/44; A61K 9/00; A61P 43/00

(52) U.S. Cl. CPC A61K 33/44; A61K 9/0053; A61P 43/00

(57) ABSTRACT

Veterinary compositions and methods employing activated charcoal optimized for adsorption and removal of toxins from animals are disclosed. Formulations include oral suspensions, pastes, boluses, feed additives, and enteral-compatible suspensions with defined charcoal physical characteristics and optional adjuvants to enhance palatability, adsorption specificity, and safety. Species-specific dosing regimens and administration kits for emergency veterinary toxin management are provided.

### FIELD OF THE INVENTION

This invention relates to systems that improve palatability of activated charcoal, enhance acceptance by the animal, and enable voluntary ingestion of activated charcoal for therapeutic purposes for the removal of toxins. More specifically, the invention relates to palatability-enhanced activated charcoal systems configured to enable voluntary ingestion and early administration in non-clinical settings, thereby improving treatment outcomes for toxin exposure in animals. The invention provides flavoring and formulation systems that improve palatability of activated charcoal and enhance acceptance by the animal.

Definitions as used herein are:
“Activated charcoal” means carbonaceous materials thermally or chemically activated to provide high specific surface area and porosity suitable for adsorption of organic and inorganic toxicants.
“Animal” includes companion animals (e.g., dogs, cats), production animals (e.g., cattle, swine, poultry), equines, and exotic or small mammal species.

### BACKGROUND

Animals may be exposed to a wide variety of toxicants including pharmaceutical agents, plant toxins, mycotoxins, heavy metals, food not suitable for ingestion (ie chocolate, grapes etc), and chemical contaminants. Activated charcoal (also called activated carbon) is known as an adsorbent used in emergency management of poisoning due to its high surface area and porosity. However, existing activated charcoal products are poorly suited for voluntary ingestion due to their unpalatable taste, messy physical characteristics, and the need for forced administration, for instance, syringe feeding or gastric intubation, which limits their use primarily to veterinary clinical settings. Existing uses of activated charcoal in veterinary medicine are limited by issues of palatability, dosing accuracy, variable adsorption in different gastrointestinal environments, and species-specific safety concerns. As a result, early administration—critical for effective toxin binding—is often delayed due to transport time to veterinary facilities and the difficulty of administering charcoal in a home setting. There is a need for veterinary-specific activated charcoal compositions and administration methods that optimize adsorption efficacy while improving ease of administration and safety across species. There is a particular unmet need for systems that enable voluntary ingestion by animals, allowing for rapid, at-home intervention during the critical early window following toxin exposure. Toxic substances can cause severe harm to the animal's health, and in some cases, even death. Because many drugs and toxins ingested by animals at home do not have specific antidote for the treatment of poisoning, gastric decontamination techniques are employed to treat overdoses or poisoning by preventing the further absorption of drug. The timely detoxification of these animals is critical to their survival, and the existing methods have limitations.

### SUMMARY OF THE INVENTION

The present invention relates to veterinary formulations and methods employing activated charcoal for adsorption and removal of toxins from animals. In particular, the invention provides a palatability-enhancing system that enables voluntary ingestion of activated charcoal, eliminating or reducing the need for forced administration and enabling earlier delivery following toxin exposure.

The invention further provides compositions, delivery systems, and kits designed for use in non-clinical environments, including household settings, to facilitate rapid first-aid intervention. More particularly, the invention concerns compositions, dosing regimens, delivery forms, and adjuvants optimized for use in companion, production, equine, and exotic animal species.

Some embodiments of the invention include a method to detoxification and gastrointestinal decontamination comprising: dosing an amount of activated carbon compounds; and providing the dosed amount of activated carbon compounds to the animal. In certain embodiments, the activated carbon compounds are configured to be voluntarily ingested by the animal without the need for restraint or forced administration.

In some embodiments, the animal has consumed or is suspected of consuming or having consumed a toxic substance. In some embodiments, administration occurs within minutes to hours of suspected ingestion in a non-clinical setting.

Some embodiments further comprise identifying the animal based on one or more of behavioral, symptomatic, and evidentiary information.

In some embodiments, the behavioral information includes one or more of: whining, pacing, panting, and crying; the symptomatic information includes one or more of: pain, vomiting, diarrhea, and constipation; and the evidentiary information includes one or more of: spilled substances, partially eaten items, and empty wrappers.

In some embodiments, dosing an amount of activated carbon compounds comprises measuring an amount of activated carbon compounds to provide to the animal.

In some embodiments, the substance is selected from: food, drink, peanut butter, canned food, water, broth, and softened food.

In some embodiments, providing the dosed amount of activated carbon compounds comprises forcibly providing the dosed amount to the animal by gastric intubation or gavage.

Some embodiments further comprise monitoring the animal for changes in behavior or symptoms.

Some embodiments further comprise: if symptoms or behavior do not improve or if symptoms or behavior worsen, dosing a second amount of activated carbon compounds; and providing the second dosed amount of activated carbon compounds to the animal.

Some embodiments include a composition for detoxification, comprising a container including an amount of activated carbon compounds. In some embodiments, the composition further comprises a palatability-enhancing system configured to maintain flavor effectiveness despite the adsorptive properties of activated charcoal.

In some embodiments, the activated carbon compounds comprise a flavored activated-charcoal composition. In certain embodiments, the palatability-enhancing system is resistant to adsorption by the activated charcoal, thereby preserving flavor and enabling voluntary ingestion.

Some embodiments further comprise an edible ingredient, in which the activated carbon compounds are mixed.

In some embodiments, the edible flavor ingredient is selected from the group consisting of: a dog biscuit, a wet treat, a gelatinous treat, a cat treat, a human food, a snack, meat, poultry, seafood, dairy, nut, fruit, vegetable, herbal, or other flavor types.

In some embodiments, the activated charcoal contains optional adsorptive materials such as kaolin or bentonite.

In some embodiments, the activated charcoal can be prepared as a gel, paste, powder, capsule, or liquid to facilitate oral administration to animals. Gel and paste formulations may be preferred due to higher acceptance rates and improved ease of administration in voluntary ingestion scenarios.

Other embodiments include:
Activated charcoal compositions with defined physical properties (e.g., surface area, pore distribution, particle size) formulated as oral suspensions, pastes, gels, boluses, feed additives, or nasogastric-compatible suspensions.
Use of adjuvants and excipients to enhance palatability, stabilize suspensions, improve adsorption for selected toxin classes, and mitigate adverse effects.
Species-specific dosing regimens, single- and multiple-dose protocols, and co-administration guidance with emetics, cathartics, fluids, and antidotes.
Systems configured to improve compliance, reduce stress to the animal and caregiver, and eliminate the need for forced administration.
Pre-measured dosing systems and weight-based administration formats to improve dosing accuracy in non-clinical settings.
Emergency first-aid kits designed for rapid deployment immediately following suspected toxin exposure.
Kits containing pre-measured single-dose units and administration implements for field or clinic use.

Preferred activated charcoal properties include, but are not limited to:
Specific surface area: approximately 500 to 2500 m2/g (preferred 800 to 1800 m2/g).
Pore size distribution: combination of micropores and mesopores to permit adsorption of small-molecule to larger organic toxins.
Particle size distribution: powder form ~10-500 μm; agglomerated/aggregate form for boluses ~0.5-5 mm.
Moisture content and ash content controlled for stability and safety. These parameters may be selected to balance adsorption capacity, flow properties, suspension stability, and species-specific safety.

Formulations and Excipients Representative formulation types include:
Oral Suspensions: activated charcoal powder dispersed in an aqueous vehicle (sterile or potable water) with viscosity agents such as xanthan gum or carboxymethylcellulose, concentration examples 100-500 mg/mL. Flavoring or sweetening agents may be included to enhance palatability.
Pastes/Gels: concentrated charcoal in gel or paste bases (e.g., carboxymethylcellulose, polyethylene glycol) with humectants and flavoring agents for direct oral administration or administration via dosing applicator.
Boluses/Tablets: compressed or molded aggregates of activated charcoal optionally coated with taste-masking or controlled-disintegration layers; aggregate size suitable for species-specific swallowing.
Feed-Blend Additives: dry charcoal blended with carrier materials, flow aids, and binders for incorporation into feed rations.
Nasogastric/Enteral-Compatible Suspensions: lower-viscosity suspensions formulated to pass through enteral tubes.

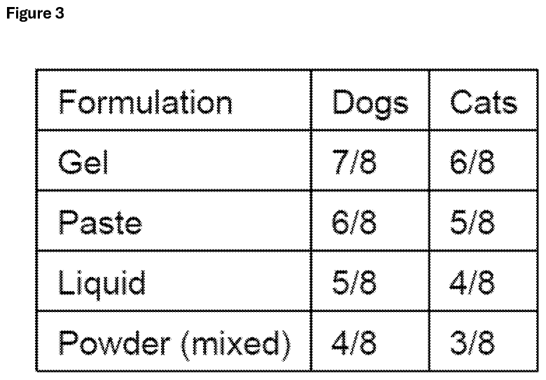

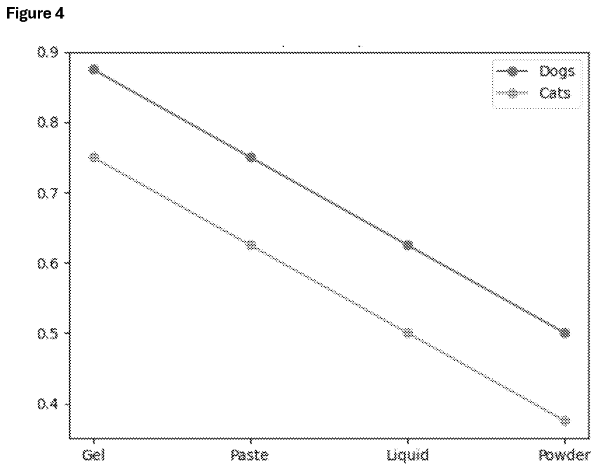

The composition may be provided as gel, paste, liquid, suspension, or powder. Powder may be mixed with food or liquid prior to administration. Palatability-enhancing systems improve acceptance across all forms. Gel-based formulations showed the highest acceptance, followed by paste, liquid, and powder, as illustrated in FIG. 3 and FIG. 4.

Excipients and adjuvants may include:
Binders and flow agents: microcrystalline cellulose, starches, colloidal silica.
Viscosity modifiers: xanthan gum, carboxymethylcellulose, gelatin.
Palatants and flavorings: species-appropriate flavoring agents and sweeteners.
Sorbent enhancers: ionic surfactants, polymeric affinity modifiers, or functional coatings designed to increase affinity for specific toxin classes (e.g., lipophilic compounds).
Cathartics/laxatives: sorbitol, polyethylene glycol, or other agents optionally included in controlled amounts to reduce gastrointestinal residence time of adsorbed toxins.
Rehydration/electrolyte components: electrolytes and buffers for animals at risk of dehydration or acid-base imbalances.

Other features and advantages of the present invention will become apparent from the following detailed description.

### DETAILED DESCRIPTION

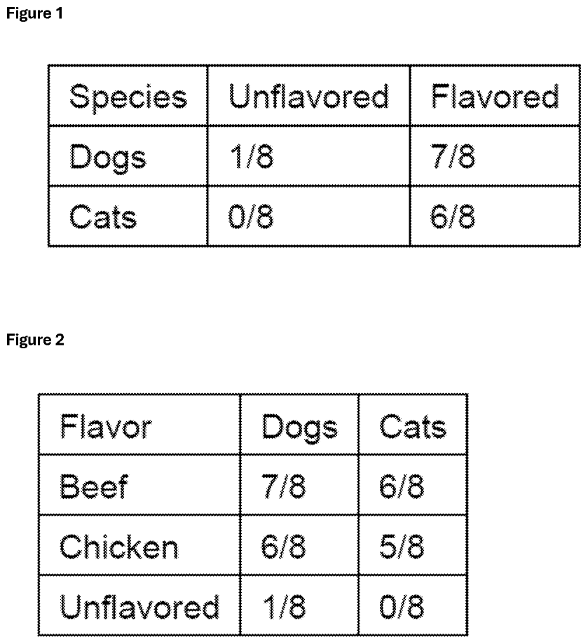

Toxicity can come from any number of avenues including accidental and intentional ingestion. Common intoxicants and/or toxic agents, in veterinary medicine, include (but are not limited to) prescription and over-the-counter medications, veterinary medications, health supplements, contaminated foods, household goods (e.g., cleaning agents, insecticides, pesticides, rodenticides, herbicides, etc.), poisonous plants, and outdoor or industrial products (e.g., antifreeze, ethylene glycol, gasoline, paint thinner, etc.). For many animals, foods that are safe for human consumption may be toxic, such as grapes, raisins, chocolate, onions, among others. The process of toxin ingestion, charcoal administration, binding, and elimination is illustrated in FIG. 1.

One of the primary ways to decontaminate poisoned animals is via emesis induction such as using an emesis agent. Emesis induction is a gold standard procedure for gastric decontamination in the veterinary patient if it is deemed safe based on the toxin ingested. However, safe and effective emesis agents may not be available at home for pets such as dogs and/or cats. For example, cats should have emesis induction performed by the veterinarian with the use of alpha2-adrenergic agonists (e.g., xylazine, dexmedetomidine). In dogs, the use of hydrogen peroxide can be recommended for at-home emesis, and a centrally acting opioid such as apomorphine is used in hospital. But the appropriate toxicology history and the dosages of emesis agent should be known before performing emesis induction. Additionally, emesis induction is inconsistent in terms of gastric decontamination, as studies show that emesis attempts at home with hydrogen peroxide result in the expulsion of toxin 60% of the time; however when it does result in toxin expulsion it only results in expulsion of 45% of the toxin. These findings are similar to other findings in human and veterinary medicine that call into question the effectiveness of emesis agents for decontamination. Therefore, Induced emesis is a questionable procedure for the treatment of acute poisoning.

Activated carbon (or activated charcoal) can be used as a gastrointestinal adsorbent in animals. To be most effective, activated charcoal, should be administered as soon as possible following toxin ingestion; however, in practice, administration is frequently delayed due to logistical barriers associated with veterinary care access.

In veterinary medicine, administration shortly after intoxication is almost impossible because of driving time (to the clinic), lapsed time since ingestion, time to triage, and the amount of time it takes to physically deliver activated charcoal (e.g., syringe feeding, orogastric tube). As a result, administration of activated charcoal is often delayed up to an hour or more. In addition, limitations of activated charcoal include its high volume needed to achieve affects, its unpalatable nature, its tendency to stain all that it touches, its potential to cause gastrointestinal impaction due to its fine physical nature, and its difficulty administering to animals in a veterinary and home environment. Furthermore, activated charcoal traditionally requires coadministration with liquids due to the unpalatable nature and physical characteristics of charcoal. These characteristics that make charcoal difficult to administer and potentially unsafe in the hands of pet owners, practically confine the use of activated charcoal to the hospital setting The inventors made the inventive realization that appropriate activated charcoal products can be beneficial for home use to detoxify intoxicated pets in a timely manner. However, existing activated charcoal products are not able to be used directly for detoxification purposes due to the safety and logistical concerns discussed. There is a significant need for detoxifying agents, including activated charcoal/carbon, in a consumer-friendly, household setting, especially when it's flavored activated charcoal as in the present invention. Conventional charcoal products are difficult to administer due to poor palatability, high volume requirements, and the need for forced delivery, limiting their suitability for home use. The inventor recognized that enabling voluntary ingestion through a palatability-enhancing system allows earlier administration and improved treatment outcomes.

Many embodiments implement activated carbon for the detoxification of intoxications. Many embodiments provide methods to detoxify individuals, including mammals, research animals, domesticated animals, livestock, and household pets (e.g., dogs, cats, birds, rodents, horses, cattle, and/or sheep) from toxic substances. Some embodiments use the activated carbon compounds for detoxifying dogs and cats. Many embodiments are directed to compositions including activated carbon that can be used to detoxify toxins and other intoxications in an individual. Many embodiments of compositions can be administered orally.

In certain embodiments, over-the-counter (OTC) or first-aid formulations may utilize lower dosing ranges relative to clinical or hospital-based protocols, for example approximately 0.25-1 g/kg body weight in dogs and cats for initial administration, although such ranges are illustrative and non-limiting. These formulations are specifically configured for safe, early intervention in home or field environments and are designed to be administered through voluntary ingestion rather than forced delivery. In some embodiments, dosing may be provided as pre-measured or weight-banded amounts (e.g., small, medium, and large animals) to facilitate ease of use in non-clinical settings. These OTC formulations are configured to support early intervention following suspected toxin ingestion and may be administered through voluntary ingestion without forced delivery. Such dosing approaches are generally lower than those used in veterinary-supervised environments, where higher and repeat dosing (e.g., approximately 1-4 g/kg) may be employed based on clinical condition. The dosing ranges described herein may be adjusted based on formulation, concentration, species, and veterinary guidance, and are intended to complement, rather than replace, clinical treatment protocols. The ability to administer activated charcoal in these lower-dose, palatability-enhanced formulations enables voluntary ingestion and earlier intervention compared to conventional clinician-administered protocols

Dosing and Administration General principles:
Early administration after oral exposure increases efficacy.
Dosage and frequency may be adjusted by species, body weight, type and amount of toxin, time since ingestion, and clinical condition. Representative dosing ranges (illustrative; to be refined by clinical testing and veterinary guidance):
Dogs and cats: dose range-single dose range 0.25-4 g/kg body weight; repeat dosing every 4-6 hours if clinically indicated up to cumulative limits based on tolerance.
Horses: single-dose range ~0.5-2 g/kg body weight; typically administered as paste or via nasogastric tube.
Ruminants (cattle, sheep): single-dose range ~0.5-2 g/kg body weight, administered as drench or feed admixture.
Small mammals and exotics: scaled doses based on weight and species-specific tolerances. Co-administration: protocols may specify timing relative to emetics, antidotes, or cathartics to avoid impairing antidote absorption or causing aspiration risk.

Safety, Contraindications, and Monitoring
Do not administer activated charcoal to animals lacking airway protective reflexes unless airway protection (e.g., endotracheal tube) is provided.
Monitor for constipation, ileus, or impaction risk in species prone to gastrointestinal stasis (e.g., rabbits, horses). Adjust formulation (e.g., inclusion of laxative) accordingly.
Consider potential adsorption of simultaneously administered orally delivered antidotes or nutrients.
Ensure hydration and monitor electrolyte status when cathartics are used.

The following examples illustrate embodiments without limiting the scope of the claims.

Example 1—Preparation of Oral Suspension A 200 mg/mL activated charcoal suspension is prepared by dispersing 20 g of activated charcoal (specific surface area~1200 m2/g, particle size ~50-200 μm) in 100 mL of potable water containing 0.5% w/v xanthan gum and a species-appropriate flavorant. Suspension is homogenized and stored at ambient temperature for short-term use. In vitro adsorption testing shows>90% removal of acetaminophen from aqueous solution within 30 minutes under specified assay conditions.

Example 2—Equine Paste Formulation and Use An equine paste is prepared containing 50% w/w activated charcoal (aggregated form), 30% w/w carboxymethylcellulose gel base, and 20% w/w humectant/flavor blend. The paste is administered via nasogastric tube at an approximate dose of 1 g/kg following suspected ingestion of a toxic plant. Clinical monitoring and subsequent toxin assays indicate reduced systemic toxin levels compared to historical untreated controls.

Example 3—Canine Taste-Masked Bolus A canine bolus containing aggregated activated charcoal (2 g per bolus) is formed with a taste-masking polymeric coating. Palatability and ease-of-administration testing in a cohort of dogs demonstrates acceptance when administered with routine handling and improved dosing accuracy compared to loose powder administration.

The activated carbons are processed (or activated) to have small, low-volume pores that can greatly increase the surface area available for adsorption or chemical reactions (depending on the adsorbate). Adsorption is a physical process where atoms or molecules adhere to surfaces of a material. Activation renders the activated carbons with a high surface-area-to-volume ratio. In several embodiments, activated carbon can be incorporated into compounds. The compounds in accordance with some embodiments can include activated carbon.

In many embodiments, the activated carbon is of high purity and quality, ensuring the safety and effectiveness of the compounds. Precursors such as (but not limited to) resin matrix can be used as the basis of the activated carbon. In several embodiments, the activated carbon can be used directly as the final compounds. In some embodiments, additives can be optionally added to the activated carbon to form the final compounds. The compounds can be molded into desired shapes and sizes. Such shapes and sizes can make consumption by an individual easier.

In several embodiments, activated carbons can have various shapes and sizes to achieve optimal detoxification effectiveness in animals. In some embodiments, the activated carbons can have a shape with one or more rounded comers. The rounded corners can increase the surface area to enhance adsorption. In some embodiments, the activated carbons can be in the form of powders, granules, particles, grains, beads, spheres, crystals, ovals, cylinders, and any combinations thereof. As can be readily appreciated, the activated carbons can have any shape that contains rounded corners as appropriate to the requirements of specific applications in accordance with various embodiments of the invention.

In several embodiments, polymers such as (but not limited to) resin matrix can be used as precursors to form the activated carbon. Examples of suitable materials for the resin matrix include (but are not limited to) polyurethane, epoxy, polyester resins, and any combinations thereof. During the activation step, the polymers can be removed to form pure activated carbon.

In many embodiments, the activated carbon can be used directly as the activated carbon compounds for detoxification in animals. The dosage of the activated carbon in the compounds can be optimized as appropriate to the requirements of specific applications in accordance with various embodiments of the invention.

Several embodiments of a compound further utilize other components and excipients. For example, flavors, flavor enhancers, buffering agents, thickeners, stabilizers, and/or plasticizers to make the dosage form more palatable. In many embodiments, the components are non-reactive with the compounds to maintain efficacy of the compounds during storage. Common flavoring agents and flavor enhancers that may be included in the formulation of the present invention include, but are not limited to, fruit (apple, orange, berry), mint, chocolate, beef, chicken, bacon, tuna, peanut butter, cheese, and any other flavors. The compositions include a flavor system that remains effective despite the adsorptive properties of activated charcoal, enabling voluntary ingestion. This mechanism is illustrated in FIG. 2. In certain embodiments, the composition includes a palatability-enhancing system that maintains flavor integrity despite the adsorptive properties of activated charcoal, enabling consistent taste perception by the animal. This system allows for voluntary ingestion, representing a significant improvement over traditional activated charcoal formulations that require forced administration.

In many embodiments, the compounds can be molded into various shapes and sizes for easy intakes of animals. In some embodiments, the compounds can be molded in the form of granules, particles, grains, beads, spheres, ovals, cylinders, and any combinations thereof. In several embodiments, the compounds can be molded in the form of polyhedrons, cubes, cuboids, triangles, tetrahedrons, bone shapes, star shapes, fish shapes, bow-tie shapes, and any combinations thereof. As can be readily appreciated, the compounds can have any form as appropriate to the requirements of specific applications in accordance with various embodiments of the invention.

Certain embodiments provide activated carbon compounds as kits, products, another item of manufacture and combinations thereof. Such embodiments can include activated carbon compounds provided as aliquots measured for a specific dose or use.

Certain embodiments provide a vessel (e.g., container) containing activated carbon compounds. Some embodiments may include ampules, vials, jars, bottles, envelopes, sachets, and/or other storage vessels containing activated carbon compounds. In certain embodiments, a vessel includes a specific number of doses that can be used (e.g., 1 dose, 2 doses, 3 doses, 4 doses, 5 doses, 10 doses, etc.). In some embodiments, the doses may be based on mass of activated carbon compounds (e.g., 50 g, 100 g, 150 g, 200 g, 250 g, etc.). In some embodiments, the doses may be for a particular size of the individual to receive the dose (e.g., <15 lbs., 16-30 lbs., 31-45 lbs., 46-100 lbs., 100-150 lbs., 150-200 lbs., >200 lbs., etc.). Such embodiments may include lines, graduations, demarcations, or other marking to assist in dosing or portioning activated carbon compounds. In certain embodiments, a single vial can include markings and/or instructions for a particular dose (e.g., 1 dose, 2 doses, 15 grams, 30 grams, pets<15 lbs., pets 16-30 lbs., etc.).

In some embodiments, the products containing activated carbon compounds can be stored dry or desiccated. In some embodiments, the products containing activated carbon compounds can be stored in a medium such as (but not limited to) water, aqueous solution, ionic solution, gel. In embodiments to be consumed, the media is preferably nontoxic. Such media may include buffers, flavors, thickeners, stabilizers, plasticizers, etc. In many embodiments, the media is non-reactive with activated carbon compounds to maintain efficacy of the activated carbon compounds during storage.

Certain embodiments are packaged as treats, snacks, or other ingestible forms. Such embodiments may be easier to provide to an individual due to an increased pleasure of consuming. Some embodiments are dog treats or snacks, such as beef, bacon, peanut butter, cheese, chicken, and/or any other flavor treat or snack that includes activated carbon compounds within the snack. Exemplary snacks or treats include dry treats (e.g., dog biscuits). Further embodiments are wet or gelatinous treats or snacks, for example, a squeezable gel treat. Squeezable treats or snacks can be provided directly from a container, provided on a dish (e.g., bowl, plate, etc.), and/or mixed with food.

Certain embodiments are packaged as foods, treats, snacks, condiments, additives, beverages, or other ingestible forms for humans. Some embodiments are powders, bars, syrups, gels, sauces, beverages, and/or any other forms of food that includes activated carbon compounds.

Certain embodiments are packaged as a post-mix product, such that one container contains activated carbon compounds (dry or in media), while a second container includes a mixing agent to assist with providing or administering the activated carbon compounds to an individual. The second container may include a solid, liquid, gel, or other agent to assist. Some mixing agents can be flavored, such as described above. Various post-mix embodiments may be packaged as a multi-part envelope, similar to candies such as Fun Dip and/or Lik-M-Aid. In certain embodiments, packaging material can be biodegradable. Biodegradable packaging may be beneficial or preferable during camping, hiking, or other outdoor activities, where convenience and environmental stewardship are desirable.

Industrial Applicability The disclosed compositions and methods are applicable to veterinary emergency medicine, agricultural animal health, and field or clinic-based toxin management programs.

Closing The foregoing description and examples illustrate embodiments of the invention. Variations and modifications within the scope of the appended claims will be apparent to those skilled in the art.

### Methods of Use

In a number of embodiments, activated carbon compounds are administered to an individual (including a household pet such as dogs, cats, or other small mammals) that have ingested toxic substances. The activated carbon binds with the toxins in the digestive tract to prevent the toxins from being absorbed into the bloodstream. The bound toxins can then be eliminated from the body through excrement (e.g., feces). Adsorption of the adsorbate (toxin) to the activated carbon occurs through van der Waals forces rather than chemical binding.

In embodiments, where activated carbon compounds are pre-mixed (e.g., made into a treat or snack), dosing can include counting the amount of snacks or treats containing enough activated carbon compounds for a particular size of individual (e.g., each treat is sufficient for up to 15 pounds of mass).

In post-mix embodiments, dosing includes mixing the activated carbon compounds with a substance to ease consumption of the activated carbon compounds. Such mixing can include mixing activated charcoal with substances to ease consumption, such as an edible substance. Such substances can include certain nut butters (e.g., peanut butter, almond butter), a liquid (e.g., water, broth, beverage, etc.), wet food, softened food (e.g., dry kibble mixed with water or another liquid), canned tuna, potted meat, and/or any other edible substance.

Depending on the dose preparation, the dose activated carbon compounds can be provided to the individual for voluntary consumption, such as when the individual takes the dose of activated carbon compounds without assistance. In preferred embodiments, the composition is configured such that the animal voluntarily consumes the activated charcoal without the need for restraint, syringe feeding, or gastric intubation. This voluntary ingestion improves compliance, reduces stress, and enables earlier intervention in toxin exposure scenarios.

In certain embodiments, the dose of activated carbon compounds is forcibly provided via forced consumption, such as by gastric intubation, gavage, and/or any other relevant process to get the dose of activated carbon compounds into the stomach of the individual. Forced consumption may be necessary when the individual is unable or unwilling to consume the dose of activated carbon compounds themselves and would be performed in the veterinary setting.

The individual is monitored. Such monitoring is to determine efficacy of detoxification and can include identifying any changes in symptoms and/or behavior. Ideally, changes in the symptoms and/or behavior are to improve and/or return to normal. Monitoring can extend until such time that symptoms and/or behavior return to normal (e.g., non-intoxicated behavior).

However, if intoxication is too great, symptoms and/or behavior may not change, may worsen, or only improve slightly. In such circumstances, various embodiments may repeat dosing activated carbon compounds, providing a dose of activated carbon compounds, and monitoring the individual until symptoms and/or behavior improve and/or return to normal. Repeat dosing can occur at various intervals as appropriate for size of the individual and/or severity of intoxication.

1. A veterinary composition for reducing systemic absorption of an ingested toxin in an animal, comprising:
(a) activated charcoal; and
(b) a palatability-enhancing system comprising one or more flavoring agents, carriers, coatings, or encapsulating components, wherein the palatability-enhancing system is configured to maintain detectable flavor perception in the presence of the activated charcoal despite adsorptive properties of the activated charcoal and to enable voluntary oral ingestion by the animal without restraint or forced administration.

2. The composition of claim 1, wherein the palatability-enhancing system maintains detectable flavor perception after contact with activated charcoal.

3. The composition of claim 1, wherein the palatability-enhancing system comprises a flavor component that is physically or chemically protected from adsorption by the activated charcoal.

4. The composition of claim 2, wherein the palatability-enhancing system maintains flavor perception after mixing with activated charcoal in vivo.

5. The composition of claim 3, wherein the flavor component is encapsulated, lipid-bound, polymer-bound, or coated.

6. The composition of claim 1, wherein the composition is voluntarily consumed without syringe feeding, restraint, or gastric intubation.

7. The composition of claim 1, wherein the palatability-enhancing system improves voluntary consumption relative to unflavored activated charcoal.

8. The composition of claim 1, wherein the composition is configured for administration in a non-clinical environment following toxin ingestion.

9. A veterinary composition for reducing systemic absorption of an ingested toxin in an animal, comprising activated charcoal having a specific surface area of 500 to 2500 m2/g and a particle size distribution of 10 to 500 micrometers, dispersed in a pharmaceutically acceptable oral carrier.

10. The composition of claim 1 formulated as an oral paste or gel comprising a humectant and a flavoring agent.

11. The composition of claim 1 formulated as a bolus comprising aggregated activated charcoal particles having a diameter of 0.5 to 5 mm and a taste-masking coating.

12. The composition of claim 1 further comprising an adsorbent-enhancing adjuvant selected from ionic surfactants and polymeric affinity modifiers configured to increase adsorption of lipophilic toxins.

13. A method of treating an animal exposed to a toxin, comprising administering to the animal an effective amount of the composition of claim 1 within 4 hours of exposure.

14. The method of claim 13, wherein the effective amount is 0.25 to 4 g/kg body weight for dogs and cats.

15. The method of claim 13, further comprising co-administering a veterinarian-selected cathartic or antidote, and repeating administration of the composition every 4 to 6 hours based on clinical judgment.

16. The method of claim 13, wherein the effective amount is 0.25 to 1 g/kg body weight.

17. A kit for veterinary toxin management comprising: a supply of the composition of claim 1 in pre-measured single-dose units; instructions identifying species-specific dosing; and an administration implement suitable for enteral dosing.

18. A method of reducing systemic absorption of an ingested toxin in an animal, comprising administering activated charcoal having a specific surface area of 500 to 2500 m²/g and a particle size distribution of 10 to 500 micrometers.

19. The composition of claim 1, wherein the palatability-enhancing system is resistant to adsorption by the activated charcoal.

20. The method of claim 13, wherein administration occurs in a non-clinical setting within 4 hours of suspected ingestion.

21. A veterinary toxin management kit comprising:
(a) the composition of claim 1 in pre-measured doses;
(b) instructions for species-specific dosing; and
(c) an administration implement.

22. The composition of claim 1, wherein the composition is formulated as a gel, paste, liquid, suspension, bolus, or edible treat.

23. The composition of claim 1, wherein the composition is packaged in a weight-based dosing format for consumer use.

24. The composition of claim 1, wherein the animal is selected from a companion animal, livestock animal, or equine.
